(19) United States
(12) Patent Application Publication
(10) Pub. No.: US 2014/0121178 A1
(43) Pub. Date: May 1, 2014

(54) METHOD AND IMPROVED PHARMACEUTICAL COMPOSITION FOR IMPROVING THE ABSORPTION OF AN ESTER PRODRUG

(71) Applicant: Haiyung Cheng, Coral Springs, FL (US)

(72) Inventor: Haiyung Cheng, Coral Springs, FL (US)

(73) Assignee: ACENDA PHARMA INC., Coral Springs, FL (US)

(21) Appl. No.: 14/129,263

(22) PCT Filed: Jun. 24, 2011
(86) PCT No.: PCT/CN2011/076256

§ 371 Date: Dec. 24, 2013

(51) Int. Cl. A61K 47/14; A61K 45/06; A61K 31/7068; A61K 31/4178; A61K 31/4365

(52) U.S. Cl. CPC A61K 47/14; A61K 31/4178; A61K 31/4365; A61K 31/7068; A61K 45/06
USPC 514/49; 514/382; 514/301; 514/338; 514/283

(57) ABSTRACT

The present invention relates to a method and an improved composition for improving the absorption of an ester prodrug in a subject. The method includes co-administering to the subject an effective amount of the ester prodrug or a pharmaceutical acceptable salt thereof, and a sufficient amount of adjuvant to impede a carboxylesterase-mediated hydrolysis of the ester prodrug in vivo, wherein the adjuvant is selected from the gnzup consisting of triacetin, triethyl citrate and a combination of both. The present invention also relates to a method for impeding carboxylesterase-mediated hydrolysis of esters, including ester prodrugs.

### CROSS-REFERENCE TO RELATED APPLICATION

This application is a U.S. National Stage of International Application No. PCT/CN2011/076256, filed on Jun. 24, 2011, for which priority is claimed under 35 U.S.C. §120, the entire contents of which are hereby incorporated by reference.

### BACKGROUND OF THE INVENTION

1. Field of the Invention

The present disclosure relates to a method and an improved composition for improving the absorption of ester prodrugs; and a method for impeding carboxylesterase-mediated hydrolysis of esters, including ester prodrugs. More particularly, the present disclosure relates to a method and an improved composition for improving the absorption of ester prodrugs by use of an adjuvant that impedes carboxylesterase-mediated hydrolysis of ester prodrugs.

2. Description of Related Art

Almost all drugs are metabolized by cytochrome P450 (CYP) isozymes. Some drugs are rapidly degraded by esterase enzymes in the gastrointestinal (GI) tract, liver and/or central circulation before arriving at their target sites or reaching certain levels in the central circulation to confer therapeutic effect. Two pharmacokinetic parameters, i.e., the area under the plasma concentration versus time curve (AUC) and peak plasma concentration (Cₘₐₓ) are commonly used to assess the absorption pharmacokinetics of a drug. The degree of improvement of absorption kinetics and the availability of a drug in the central circulation (i.e., bioavailability), however, are assessed mainly by using AUC of the drug. The absorption kinetics and oral bioavailability of some drugs, which are therapeutically active but are poorly absorbed, can be improved by the synthesis of their ester prodrugs which are more readily absorbed from the GI tract. Ester prodrugs are prodrugs having ester moieties. Once absorbed, ester prodrugs undergo hydrolysis to generate active drugs under the action of the esterase. Thus, the AUC and Cₘₐₓ values of prodrugs and/or their active drugs are commonly used to assess the absorption kinetics and oral bioavailability of ester prodrugs.

Esterase is a hydrolase enzyme which catalyzes the hydrolysis of an ester into its acid and alcohol. Esterase activity is found in various human tissues and organs including the liver, GI track, kidney, large intestine, lung, placenta, skeletal muscles, uterus, heart, and blood. As such, some ester prodrugs undergo premature hydrolysis in the GI tract even before it can be absorbed or premature hydrolysis in the liver after intestinal absorption, leading to poor oral bioavailability and the need for more frequent and higher doses than are most desirable. In particular, the majority of intestinal esterase is present in the absorption sites of small intestine thereby offsetting the increased efficiency of prodrugs to pass the intestinal barrier. Thus, methods and compositions are sought and a number of approaches have been tried to overcome this problem and to improve the absorption kinetics and, thus, to enhance the oral bioavailability of ester prodrugs.

For example, esterase inhibitors including organophosphates (e.g., p-nitrophenyl phosphate), carbamates (e.g., neostigmine), p-hydroxymercuribenzoate, derivatives of nitrophenol and sulfonamide, trifluoromethyl ketones, benzil, isatins (or 1H-indole-2,3-dione), and aryl ureas have been utilized or synthesized to inhibit the esterase-mediated hydrolysis of ester prodrugs. However, organophosphates, carbamates, and p-hydroxymercuribenzoate are regarded as highly toxic poisons.

Another approach utilizes formulations containing substrates for the esterase to impede esterase-mediated hydrolysis of ester prodrugs; examples of substrates include fruit extracts and phospholipids such as lecithin. Among a multitude of other substances, fruit extracts contain several flavoring esters. It is postulated that inhibition of the metabolism of the drug by these esters and/or by other compounds could at least partially explain the absorption enhancement observed in the presence of the fruit extract. However, it is not feasible to incorporate the fruit extract in a pharmaceutical formulation, since it contains a broad variety of other compounds other than the esters, which makes it difficult to control its absorption enhancing effect. In view of this, Gelder et al. investigated the effect of discrete esters and ester mixtures on the intestinal stability and absorption of tenofovir disoproxil fumarate (tenofovir DF, an esterase-sensitive prodrug of the antiviral tenofovir). However, their research indicated that the extent of inhibition of the enzymatic conversion of the prodrug to the monoester varies from one ester to another. Specifically, the effect of discrete esters on metabolism of tenofovir DF ranged from a negligible effect to almost complete inhibition (See, J. van Gelder et al., Drug Metabolism and Disposition, 2002, 30:924-930.)

In view of the foregoing, there still exists in this art a need of suitable esters, which are not only safe to use in a live subject, but may also impede enzymatic conversion of ester prodrugs in vivo; such esters are suitable substrates for human esterase, hence may act as a pharmacological adjuvant of ester prodrugs in vivo to improve absorption kinetics of the ester prodrugs. Accordingly, improved methods and pharmaceutical compositions that increase absorption and, thus, enhance the oral bioavailability of ester prodrugs, would represent a significant advancement in the art.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

The present invention is based on the unexpected discovery that when triacetin, triethyl citrate, or a combination of both is co-administered with an ester prodrug susceptible to carboxylesterase 1 (CE1)-mediated and/or carboxylesterase 2 (CE2)-mediated hydrolysis, hydrolysis of the ester prodrug is greatly impeded. Surprisingly, it is discovered that well known pharmaceutical excipients including triacetin and triethyl citrate, when applied alone or in combination, retards CE-mediated hydrolysis of ester prodrugs such as olmesartan medoxomil and clopidogrel more than lecithin does. In view of this discovery, one can use triacetin, triethyl citrate, or a combination of both to improve absorption kinetics and, thus, enhance the oral bioavailability of an ester prodrug. The ester prodrug may belong to any therapeutic class, including anti-thrombogenic agents, peroxisome proliferator-activated receptor alpha (PPARα) agonists, HMG-CoA reductase inhibitors (or statins), angiotensin II (AII) antagonists, angiotensin-converting enzyme (ACE) inhibitors, anti-coagulant, antibiotics, reverse transcriptase inhibitors, mitotic inhibitors, topoisomerase 1 inhibitors, DNA synthesis inhibitors, neuraminidase inhibitors, immunosuppressants, chemotherapy agents, gamma-aminobutyric acid (GABA) analogues, and GABA_{B} receptor agonists.

In one aspect, the present disclosure is directed to a method for improving the absorption of an ester prodrug in a subject. According to embodiments of the present disclosure, the method comprises the step of co-administering to the subject an effective amount of the ester prodrug or a pharmaceutical acceptable salt thereof, and an adjuvant selected from the group consisting of triacetin, triethyl citrate and a combination of both, wherein the adjuvant is administered in an amount sufficient to improve the absorption kinetics and, thus, enhance the bioavailability of the ester prodrug (i.e., to increase the AUC values of the ester prodrug and/or the active drug).

In another aspect, the present disclosure is directed to an improved pharmaceutical composition for improving absorption of an ester prodrug in a subject. The improved pharmaceutical composition comprises an effective amount of an ester prodrug or a pharmaceutically acceptable salt thereof and a pharmaceutically acceptable excipient; and the improvement according to embodiments of the present disclosure comprises an adjuvant selected from the group consisting of triacetin, triethyl citrate and a combination of both; wherein the adjuvant is capable of impeding carboxylase-mediated hydrolysis of the ester prodrug in vivo. According to optional embodiments of the present disclosure, the improved pharmaceutical composition may further comprise a second ester prodrug and/or additional components such as other pharmaceutically acceptable carriers, adjuvants, and vehicles thereof as desired.

Also within the scope of the present disclosure is the use of the above-mentioned pharmaceutical composition for treating conditions such as (1) cardiovascular disease (the ester prodrug being olmesartan medoxomil, candesartan cilexetil, ramipril, delapril, trandolapril, temocapril, cilazapril, quinapril, imidapril, aspirin, clopidogrel or prasugrel), (2) hypercholesterolemia, hypertriglyceridemia or both diseases (the ester prodrug being lovastatin, simvastatin, clofibrate or fenofibrate), (3) fever and rheumatic arthritis (the ester prodrug being aspirin), (4) infections including HIV and Hepatitis B infections (the ester prodrug being cefpodoxime proxetil, cefditoren pivoxil, tenofovir disoproxil, or adefovir dipivoxil), (5) cancer (the ester prodrug being paclitaxel, docetaxel, isotaxel, irinotecan or capecitabine), (6) Influenza virus A and Influenza virus B infection (the ester prodrugs being oseltamivir or A-322278), (7) spasticity and GERD (the ester prodrug being arbaclofen placarbil), and (8) sleep loss caused by restless legs syndrome and pain associated with post-herpetic neuralgia (the ester prodrug being gabapentin enacarbil), and for manufacture of a medicament for that treatment.

Further within the scope of the present disclosure is the use of the above-mentioned pharmaceutical composition for reducing the risk of conditions such as cardiovascular disease (the ester prodrug being olmesartan medoxomil, candesartan cilexetil, ramipril, delapril, trandolapril, temocapril, cilazapril, quinapril, imidapril, lovastatin, simvastatin, clofibrate or fenofibrate), Influenza virus A and Influenza virus B infection (the ester prodrugs being oseltamivir or A-322278), organ rejection (the ester prodrug being mycophenolate mofetil), blood clots (the ester prodrug being dabigatran etexilate), and atherothrombotic events (the ester prodrug being aspirin, clopidogrel or prasugrel), and for the manufacture of a medicament for reducing that risk.

The details of many embodiments of the invention are set forth in the detailed description and the claims below. Other features, objects, and advantages of the invention will become better understood with reference to the following detailed description and the appended claims.

### DESCRIPTION

The detailed description provided below is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

For convenience, certain terms employed in the specification, examples and appended claims are collected here. Unless defined otherwise, all technical and scientific terms used herein have the same meaning as commonly understood by one of the ordinary skill in the art to which the present disclosure belongs.

The singular forms “a”, “an”, and “the” are used herein to include plural referents unless the context clearly dictates otherwise.

As used herein, the term “about” generally means within 10%, 5%, 1%, or 0.5% of a given value or range. Alternatively, the term “about” means within an acceptable standard error of the mean, when considered by one of ordinary skill in the art.

The term “prodrug”, as used herein, refers to any compound that when administered to a biological system yields the “drug” substance either as a result of spontaneous chemical reaction(s) or by enzyme catalyzed or metabolic reaction(s). “Ester prodrugs” are compounds that contain ester groups imparting the prodrug nature of the drug. For example, an ester prodrug of a compound containing a carboxyl group may be convertible by hydrolysis in vivo to the corresponding carboxylic acid.

The terms “oral bioavailability” and “bioavailability” are used interchangeably to refer to the amount or portion of an orally administered drug that reaches the systemic circulation.

As used herein, a “pharmaceutically acceptable” component is one that is suitable for use with humans and/or animals without undue adverse side effects (such as toxicity, irritation, and allergic response) commensurate with a reasonable benefit/risk ratio.

The term “effective amount” or “sufficient amount” as used herein refers to the quantity of a component which is sufficient to yield a desired therapeutic response. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the structure of the compounds or its derivatives. Effective amount may be expressed, for example, as the total mass of ester prodrug (e.g., in grams, milligrams or micrograms) or a ratio of mass of ester prodrug to body mass, e.g., as milligrams per kilogram (mg/kg).

The term “excipient” as used herein means any inert substance (such as a powder or liquid) that forms a vehicle/carrier for the ester prodrug(s) and/or adjuvant. The excipient is generally safe, non-toxic, and in a broad sense, may also include any known substance in the pharmaceutical industry useful for preparing pharmaceutical compositions such as, fillers, diluents, agglutinants, binders, lubricating agents, glidants, stabilizer, colorants, wetting agents, disintegrants, and etc.

The term “adjuvant” as used herein is defined as a substance that, when added to the pharmaceutical composition, enhances the absorption kinetics, hence, the bioavailability of the ester prodrug, while having few or none of direct therapeutically effects when given by itself.

As used in the present disclosure, the term “Cₘₐₓ” refers to the maximum concentration of an active compound or drug (e.g., clopidogrel or capecitabine) in the blood plasma, whereas the term “Tₘₐₓ” means the time to achieve the maximum plasma concentration of said active compound or drug. The term “AUC₀₋ₜ” refers to an area under the curve from time zero to the last measured time point of a measurable drug concentration.

The term “treating” as used herein refers to application or administration of triacetin, triethyl citrate or both pharmaceutical adjuvants and at least one ester prodrugs to a subject, who has a medical condition, a symptom of the condition, a disease or disorder secondary to the condition, or a predisposition toward the condition, with the purpose to partially or completely alleviate, ameliorate, relieve, delay onset of, inhibit progression of, reduce severity of, and/or reduce incidence of one or more symptoms or features of a particular disease, disorder, and/or condition. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

The practices of this invention are hereinafter described in detail with respect to a method and a pharmaceutical composition for improving the absorption kinetics and, thus, enhancing the bioavailability of an ester prodrug in a subject. Results of pharmacokinetic studies, as described herein below, show that the present pharmaceutical composition, particularly, a composition that contains an ester prodrug and an adjuvant consisting of triacetin and/or triethyl citrate, may impede the carboxylase-mediated hydrolysis of the ester prodrug in vivo thereby improving the absorption kinetics and, thus, enhancing the bioavailability of the ester prodrug in the subject.

Esterases are a group of hydrolytic enzymes occurring in multiple forms with broad substrate specificity. Carboxylesterase (CE) is the most abundant esterase in the liver and small intestine of humans, monkeys, dogs, rabbits and rats. It plays an important role in biotransformation of a variety of ester prodrugs such as anti-thrombogenic agents (e.g., aspirin, clopidogrel and prasugrel), peroxisome proliferator-activated receptor alpha (PPARα) agonists (e.g., fenofibrate and clofibrate), HMG-CoA reductase inhibitors (or statins, e.g., lovastatin and simvastatin), angiotensin II (AII) antagonists (e.g., olmesartan medoxomil and candesartan cilexetil), angiotensin-converting enzyme (ACE) inhibitors (e.g., ramipril, delapril, trandolapril, temocapril, cilazapril, quinapril and imidapril), anti-coagulants (e.g., dabigatran etexilate), antibiotics (e.g., cefpodoxime proxetil and cefditoren pivoxil), reverse transcriptase inhibitors (e.g., tenofovir disoproxil and adefovir dipivoxil), mitotic inhibitors (e.g., paclitaxel, docetaxel and isotaxel), DNA synthesis inhibitors (e.g., capecitabine), topoisomerase 1 inhibitors (e.g., irinotecan), neuraminidase inhibitors (e.g., oseltamivir and A-322278), immunosuppressants (e.g., mycophenolate mofetil), gamma-aminobutyric acid (GABA) analogues (e.g., gabapentin enacarbil), and GABA_{B} receptor agonists (e.g., arbaclofen placarbil).

In humans and laboratory animals, the majority of CE isozymes belong to the carboxylesterase 1 (CE1) and carboxylesterase 2 (CE2) families. The liver contains both CE1 and CE2 isozymes in all these species. In human liver, the CE1 level exceeds the CE2 level. The human and rat small intestines contain only CE2 isozymes, while in rabbits and monkeys, both CE1 and CE2 isozymes are present. Therefore, bioconversion rates of orally administered prodrugs are affected by expression levels of CE1 and CE2 in human liver and small intestine. Although human CE1 and CE2 have overlapping substrate recognition, clear evidence of ester-based substrate specificity has been observed. Two products, an alcohol and an acyl moiety, are generated from ester hydrolysis. In general, human CE1 prefers substrates with a large acyl moiety, whereas human CE2 prefers substrates with a large alcohol group. For example, prodrugs with a large acyl moiety such as oseltamivir, clopidogrel, lovastatin, temocapril, trandolapril, cilazapril, quinapril, delapril, and imidapril are hydrolyzed predominately or solely by human CE1, whereas prodrugs with a large alcohol group such as aspirin, prasugrel, arbaclofen placarbil, and gabapentin enacarbil are hydrolyzed mainly by human CE2. Based on this substrate specificity, it can be predicted that fenofibrate, clofibrate, ramipril, A-322278, and simvastatin are the preferred substrates for human CE1, while olmesartan medoxomil, candesartan cilexetil, tenofovir disoproxil, mycophenolate mofetil, adefovir dipivoxil, cefpodoxime proxetil, cefditoren pivoxil, and isotaxel are the preferred substrates for human CE2. Moreover, it can be concluded that, besides expression levels of CE1 and CE2 in humans, bioconversion of ester prodrugs is also affected by the substrate specificity of human CE1 and CE2. Thus, one purpose of the present disclosure is to provide a compound or composition which can retard not only CE1-mediated hydrolysis but also CE2-mediated hydrolyses of ester prodrugs to improve the absorption kinetics and, thus, enhance oral bioavailability of these prodrugs.

Triacetin is affirmed as a generally recognized as safe (GRAS) human food additive by the Food and Drug Administration (FDA, USA). It is also used in pharmaceutical industry as an excipient, such as a humectant, a plasticizer, and a solvent. Likewise, triethyl citrate is commonly used as a food additive and in pharmaceutical coatings. Triethyl citrate has also been used to stabilize E-type prostaglandin compounds and to prevent lipase hydrolysis of triglycerides. Both triacetin and triethyl citrate are safe to be used in animals including human, and up to 10 mg/Kg body weight may be used in man without exerting any toxicity. In the present disclosure, triacetin and triethyl citrate are evaluated and compared to lecithin in terms of their effectiveness in impeding CE-mediated hydrolysis of ester prodrugs.

According to embodiments of the present disclosure, the improved pharmaceutical composition comprises an effective amount of an ester prodrug or a pharmaceutically acceptable salt thereof, and a pharmaceutically acceptable excipient; and the improvement comprises an adjuvant selected from the group consisting of triacetin, triethyl citrate and a combination of both; wherein the adjuvant is present in an amount sufficient to impede carboxylase-mediated hydrolysis of the ester prodrug in vivo.

In optional embodiments of the present disclosure, the improved pharmaceutical composition may further comprise a second ester prodrug and/or additional components such as other pharmaceutically acceptable carriers, adjuvants, and vehicles thereof as desired.

According to embodiments of the present disclosure, the method for improving absorption of ester prodrugs comprises administering the improved pharmaceutical composition disclosed herein to a subject. Specifically, the method comprises the step of co-administering to the subject an effective amount of the ester prodrug or a pharmaceutical acceptable salt thereof; and a sufficient amount of an adjuvant selected from the group consisting of triacetin, triethyl citrate and a combination of both, to impede carboxylase-mediated hydrolysis of the ester prodrug in vivo and thereby improves the absorption kinetics and, thus, enhance the bioavailability of the ester prodrug.

Test results summarized hereinbelow evidence that triacetin, triethyl citrate, and a combination of both of these pharmaceutical adjuvants, when used with one or two ester prodrug(s), impede CE-mediated hydrolysis of the prodrug(s). Thus, this co-administering of ester prodrugs with adjuvants identified by this invention results in an improvement in absorption kinetics (i.e., to increase the AUC values of the ester prodrug(s) and/or the active drug(s) of the ester prodrug(s)) and, thus, an enhancement in oral bioavailability of the ester prodrug(s).

According to various embodiments of the present disclosure, suitable ester prodrugs include those exemplified hereinabove and any other known or future ester prodrugs, as long as the absorption kinetics may be improved and, thus, bioavailability of such ester prodrug may be increased by the present method and/or improved composition.

Ester prodrugs used to practice the present disclosure are either commercially available or can be readily prepared by methods well known in the art. These prodrugs may occur as racemic mixtures, single enantiomers, individual diastereomers, diastereomeric mixtures, and cis- or trans-isomeric forms. Additionally, their pharmaceutically acceptable salts are also within the scope of the present disclosure. Such salts can be formed between a positively charged ionic group in a therapeutic agent (e.g., ammonium) and a negatively charged counterion (e.g., acetate, citrate, aspartate, benzoate, fumarate, chloride, bromide, lactate, maleate, oxalate, phosphate, succinate, sulfate, or tartrate). Likewise, a negatively charged ionic group in a therapeutic agent (e.g., carboxylate) can also form a salt with a positively charged counter ion (e.g., sodium, potassium, calcium, or magnesium). Non-exhaustive examples of acids which may be employed to form pharmaceutically acceptable acid addition salts include such inorganic acids as sulfuric acid, hydrochloric acid, and phosphoric acid and such organic acids as oxalic acid, maleic acid, and succinic acid. For example, clopidogrel also refers to its corresponding bisulfate salt.

In embodying the present disclosure, ester prodrug(s) and the pharmaceutically acceptable adjuvant(s) may be administered orally. A composition for oral administration may be any orally acceptable dosage form including capsules, tablets, emulsions and aqueous suspensions, dispersions, and solutions. In the case of tablets, commonly used carriers include, but are not limited to, lactose and corn starch. Lubricating agents, such as magnesium stearate, are also typically added. Tablets can additionally be prepared with enteric coatings. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

The optimal amount in a given dosage form or formulation can be estimated or determined by experimentation such as that described in the examples of this application. As shown in these examples, the amount of triacetin, triethyl citrate or a combination of both in oral dosing solutions is in the range of about 10-90% by weight. Thus, the amount of triacetin, triethyl citrate or a combination of both in an orally acceptable dosage form is generally ranged, for example, from about 1% to about 99.9% by weight; and preferably from about 10% to about 90% by weight. The examples also show that the ratio of the amount of prodrug drug to the amount of adjuvant (triacetin and/or triethyl citrate) is in the range of about 1:3-1:27. Thus, the ratio of the amount of prodrug drug to the amount of adjuvant (triacetin and/or triethyl citrate) in an oral dosage form is generally ranged, for example, from about 1:1 to about 1:50, and preferably from about 1:2 to about 1:40.

In optional embodiments where two ester prodrugs are administered in combination, the two ester prodrugs can be formulated as a single composition or separate compositions.

Some of the ester prodrugs mentioned above (i.e., olmesartan medoxomil and candesartan cilexetil) are AII antagonists and some (i.e., ramipril, delapril, trandolapril, temocapril, cilazapril, quinapril and imidapril) are ACE inhibitors. All antagonists and ACE inhibitors and their combinations are commonly used to treat cardiovascular disease (e.g., hypertension and heart failure). Thus, also within the scope of the present disclosure is a method of treating cardiovascular disease using an AII antagonist, an ACE inhibitor, or both therapeutic agents with an adjuvant identified in the present disclosure, which is triacetin, triethyl citrate, or a combination of both.

Some of the ester prodrugs (i.e., aspirin, clopidogrel, and prasugrel) are anti-thrombogenic agents. Anti-thrombogenic agents are commonly used to inhibit blood clots in coronary artery disease, peripheral vascular disease, and cerebrovascular disease. Aspirin is also commonly used to reduce fever and treat rheumatic arthritis. Some of the ester prodrugs (i.e., dabigatran etexilate) are anti-coagulants. Anti-coagulants are commonly used to prevent formation of blood clots in the veins after knee or hip replacement surgery. Thus, also within the scope of the present disclosure is a method of inhibiting blood clots using an anti-thrombogenic agent or anti-coagulant with triacetin, triethyl citrate or both; or reducing fever and treating rheumatic arthritis using aspirin with triacetin, triethyl citrate, or a combination of both.

Some of the ester prodrugs (i.e., fenofibrate and clofibrate) are PPARα agonists. Some are statins (e.g., lovastatin and simvastatin). Statins are commonly used to treat hypercholesterolemia. PPARα agonists are commonly used alone or in conjunction with statins in the treatment of hypercholesterolemia and hypertriglyceridemia. Thus, also within the scope of the present disclosure is a method of treating hypercholesterolemia, hypertriglyceridemia, or both diseases using a PPARα agonist, a stain, or both therapeutic agents with triacetin, triethyl citrate, or a combination of both.

Some of the ester prodrugs (i.e., cefpodoxime proxetil and cefditoren pivoxil) are antibiotics. Antibiotics are commonly used to treat infections. Thus, also within the scope of the present disclosure is a method of treating infections using an antibiotic with triacetin, triethyl citrate, or a combination of both.

Some of the prodrugs (e.g., oseltamivir and A-322278) are neuraminidase inhibitors. Neuraminidase inhibitors are commonly used to treat Influenza virus A and Influenza virus B infection. Thus, also within the scope of the present disclosure is a method of treating Influenza virus A and Influenza virus B infection using a neuraminidase inhibitor with triacetin, triethyl citrate, or a combination of both.

Some of the ester prodrugs (i.e., tenofovir disoproxil, adefovir dipivoxil, arbaclofen placarbil, and gabapentin enacarbil) are reverse transcriptase inhibitors, GABAB receptor agonist, and GABA analogue, respectively. They are commonly used to treat HIV infection, Hepatitis B infection, spasticity, and GERD, sleep loss caused by restless legs syndrome and pain associated with post-herpetic neuralgia, respectively. One of the ester prodrugs (i.e., mycophenolate mofetil) is an immunosuppressant and used to prevent rejection in organ transplantation. Thus, also within the scope of the present disclosure is a method of treating the above diseases using these ester prodrugs with triacetin, triethyl citrate, or a combination of both.

Some of the ester prodrugs (i.e., paclitaxel, isotaxel, docetaxel, irinotecan, and capecitabine) are mitotic inhibitors, topoisomerase 1 inhibitor, and DNA synthesis inhibitor, respectively. They are commonly used to treat cancer. Thus, also within the scope of the present disclosure is a method of treating cancer using these ester prodrugs with triacetin, triethyl citrate, or a combination of both.

In preferred embodiments of the present disclosure, the prodrug is clopidogrel, olmesartan medoxomil, tenofovir disoproxil, adefovir dipivoxil, mycophenolate mofetil, paclitaxel, docetaxel, isotaxel, irinotecan, capecitabine, arbaclofen placarbil, or gabapentin enacarbil.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

Effectiveness of a pharmaceutical adjuvant such as triacetin or triethyl citrate in impeding hydrolysis of an ester prodrug by CE can be preliminarily screened by an in vitro assay. For example, a mixture of triacetin or triethyl citrate is incubated with an ester prodrug (e.g., olmesartan medoxomil) in the presence of CE, and the concentration of prodrug in the mixture after the incubation is then compared with that of a blank control containing neither triacetin nor triethyl citrate. See Example 1 below. In Example 1, the respective effect of triacetin, triethyl citrate, and lecithin in impeding hydrolysis of olmesartan medoxomil by CE was also documented. Further, the effect of triacetin and triethyl citrate in impeding CE-mediated hydrolysis of clopidogrel was also observed. See Example 2 below. Moreover, as shown in Examples 1 and 2, a combination of triacetin and triethyl citrate is also effective in greatly impeding CE-mediated hydrolysis of olmesartan medoxomil and clopidogrel. In vivo assays can be conducted to ascertain the effectiveness of triacetin, triethyl citrate or a combination of both adjuvants in improving the absorption kinetics of ester prodrugs. See Examples 3-5 below.

### EXAMPLES

### Example 1

### Influence of Various Ester Excipients on In Vitro CE-Mediated Hydrolysis of Olmesartan Medoxomil

Various known pharmaceutical ester excipients including triacetin, glyceryl tristearate, triethyl citrate, lecithin, tri-n-butyl citrate, acetyl triethyl citrate, and acetyl tri-n-butyl citrate were respectively tested to determine whether any of them impedes hydrolysis of olmesartan medoxomil by CE in vitro. Preliminary results indicated that triacetin and triethyl citrate were promising candidates, hence further experiments were conducted below to compare the effect of triacetin, triethyl citrate or a combination of both on CE-mediated hydrolysis of olmesartan medoxomil, as compared with that of lecithin.

Test solutions (a) to (f) were prepared respectively as follows: (a) 10 μM of olmesartan medoxomil dissolved in 10% DMSO (dimethyl sulfoxide)/90% PEG400 (w/w, stability control); (b) 10 μM of olmesartan medoxomil dissolved in 10% DMSO/90% PEG400 (w/w, blank control) (c) 10 μM of olmesartan medoxomil dissolved in 10% DMSO/12% triacetin/78% PEG400 (w/w/w); (d) 10 μM of olmesartan medoxomil dissolved in 10% DMSO/12% triethyl citrate/78% PEG400 (w/w/w); (e) 10 μM of olmesartan medoxomil dissolved in 10% DMSO/12% lecithin/78% PEG400 (w/w/w); and (f) 10 μM of olmesartan medoxomil dissolved in 10% DMSO/6% triacetin/6% triethyl citrate/78% PEG400 (w/w/w/w).

CE from porcine liver (17 units/mg, available from Sigma-Aldrich) was dissolved in simulated intestinal fluid (SIF, pH=6.8) to yield a CE solution (17 units/ml). SIF was prepared by dissolving 0.6805 g of KH₂PO₄ and 0.0896 g of NaOH in 100 ml of de-ionized water.

Each 70 μl of olmesartan medoxomil solution (i.e., solutions (a)-(f)) was mixed with 70 μl of SIF and thereby forming mixtures (a) to (f). Each of the mixtures (a) to (f) was transferred to one well of a 96-well plate, and 60 μl each of the CE solution was added to respective wells containing mixtures (b) to (f) to initiate the reaction. In addition, sixty (60) μl of SIF without CE was added to mixture (a). This incubation mixture without CE was used as a stability control to determine the chemical stability of olmesartan medoxomil in the incubation mixture. The mixtures were incubated for 20 minutes under the air at 37° C. with constant shaking on a temperature-controlled heating block.

At the end of the 20-minute incubation, 100 μl of ice-cold acetonitrile was added to each well to terminate the reaction. Each mixture was vortexed and centrifuged at 15,000×g for 20 min at room temperature. The supernatants were tested in HPLC/UV analysis, in which the concentration of olmesartan medoxomil in each mixture was measured. Percentages (%) of olmesartan medoxomil remaining in the mixtures were calculated based on the obtained data. Table 1 summarizes the results of this example.

As shown in Table 1, triacetin, triethyl citrate or a combination of both, retards CE-mediated hydrolysis of olmesartan medoxomil, which is predicted to be a preferred substrate of CE2, better than lecithin.

TABLE 1
Influence of Various Ester Excipients on CE-Mediated
Hydrolysis of Olmesartan Medoxomil.

| | % Remaining of | |
|---|---|---|
| | Olmesartan Medoxomil | |
| Olmesartan Medoxomil Solution | 0 min | 20 min |
| (a) 10% DMSO + 90% PEG400 (Without CE) | 100 | 94.2 |
| (b) 10% DMSO + 90% PEG400 (With CE) | 100 | 69.4 |
| (c) 10% DMSO + 12% Triacetin + 78% PEG400 | 100 | 87.4 |
| (With CE) | | |
| (d) 10% DMSO + 12% Triethyl citrate + 78% | 100 | 82.2 |
| PEG400 (With CE) | | |
| (e) 10% DMSO + 12% Lecithin + 78% PEG400 | 100 | 74.9 |
| (With CE) | | |
| (f) 10% DMSO + 6% Triacetin + 6% Triethyl | 100 | 84.9 |
| Citrate + 78% PEG400 (With CE) | | |

### Example 2

### Influence of Triacetin, Triethyl Citrate or Both on In Vitro CE-Mediated Hydrolysis of Clopidogrel

The ability of triacetin and triethyl citrate to impede CE-mediated hydrolysis of clopidogrel, which is a preferred substrate of CE1, is demonstrated in accordance with the in vitro test method described in Example 1. Results are summarized in Table 2. The results show that triethyl citrate, triacetin and lecithin retard CE-mediated hydrolysis of clopidogrel. Surprisingly, triethyl citrate and a combination of triethyl citrate and triacetin are more effective in impeding CE-mediated hydrolysis of clopidogrel than triacetin and lecithin.

TABLE 2
Influence of Triacetin, Triethyl Citrate or Both on
CE-Mediated Hydrolysis of Clopidogrel.

| | % Remaining | |
|---|---|---|
| | of Clopidogrel | |
| Clopidogrel Solution | 0 min | 20 min |
| (a) 10% DMS0 + 90% PEG400 (Without CE) | 100 | 97.8 |
| (b) 10% DMSO + 90% PEG400 (With CE) | 100 | 52.1 |
| (c) 10% DMSO + 10% Triacetin + 80% PEG400 | 100 | 74.9 |
| (With CE) | | |
| (d) 10% DMSO + 10% Triethyl citrate + 80% PEG400 | 100 | 89.1 |
| (With CE) | | |
| (e) 10% DMSO + 10% Lecithin + 80% PEG400 | 100 | 79.2 |
| (With CE) | | |
| (f) 10% DMSO + 5% Triacetin + 5% Triethyl | 100 | 86.9 |
| Citrate + 80% PEG400 (With CE) | | |

### Example 3

### Improvement of Absorption Kinetics of Olmesartan Medoxomil

Male rats (Sprague-Dawley, 300-400 g) were surgically implanted with jugular-vein cannulas one day prior to dosing and fasted overnight (about 18-20 hours) prior to dosing. Water was available ad libitum throughout the experiment. Dosing solutions of olmesartan medoxomil (5 mg/ml) were prepared in a vehicle of DMSO/PEG400 (10/90, v/v) or DMSO/PEG400/triacetin (10/80/10, v/v). Single oral doses of the olmesartan medoxomil and olmesartan medoxomil/triacetin were each administered separately to a group of 3 male rats by gavage. Each rat received olmesartan medoxomil (5 mg/kg) in DMSO/PEG400 (10/90, v/v) or olmesartan medoxomil (5 mg/kg) in DMSO/PEG400/triacetin (10/80/10, v/v).

Blood samples (0.15 ml/rat) were collected from each rat via the jugular-vein cannula at 0, 0.25, 0.5, 1, 2, 4, 6, 8, and 24 hours after dosing. Plasma was separated from blood cells by centrifugation and frozen at −20° C. until analysis. The concentrations of olmesartan (active drug) and olmesartan medoxomil in the plasma samples were determined by LC-MS/MS. A plasma concentration-time curve was plotted based on the obtained data. From the curve, the Cₘₐₓ and the time of maximum concentration (Tₘₐₓ) values were measured and the AUC value from t=0 hour to t=24 hours (AUC₀₋₂₄ₕᵣ) was calculated using the trapezoidal rule (see Altamn, Practical Statistics for Medical Research, CRC Press, 1991, pp. 432-433 and Khan and Reddy, Pharmaceutical and Clinical Calculations, CRC Press, 2000, pp. 235-236). As expected, olmesartan medoxomil was completely hydrolyzed to olmesartan after absorption following oral administration of olmesartan medoxomil or olmesartan medoxomil/triacetin to rats. Table 3 lists the mean (n=3) values of AUC₀₋₂₄ₕᵣ, Cₘₐₓ and Tₘₐₓ of olmesartan. The results indicate that triacetin increases AUC₀₋₂₄ₕᵣ and Cₘₐₓ of olmesartan by 64% and 108%, respectively; and thus improves the absorption kinetics of olmesartan medoxomil.

TABLE 3
Improvement of Absorption Kinetics of Olmesartan Medoxomil.

| | AUC_{0-24 hr} | Cₘₐₓ | Tₘₐₓ |
|---|---|---|---|
| Treatment | (ng · hr/ml) | (ng/ml) | (hr) |
| Olmesartan Medoxomil | 1704 | 825 | 0.5 |
| Olmesartan Medoxomil + | 2800 | 1720 | 0.5 |
| Triacetin | | | |

### Example 4

### Improvement of Absorption Kinetics of Clopidogrel Bisulfate

Clopidogrel is a prodrug that is metabolized mainly by cytochrome P450 2C19 (CYP2C19) in the liver into an active drug. Being an ester prodrug, clopidogrel is also rapidly hydrolyzed by carboxylesterase, mainly in the liver, to form an inactive carboxylic acid metabolite (clopidogrel acid). Thus, ester hydrolysis and hepatic metabolism by CYP2C19 represent two competing pathways that determine the efficiency of clopidogrel. A study similar to the olmesartan medoxomil study described above in Example 3 was conducted in rats (n=3) to ascertain the effect of triethyl citrate in enhancing the oral bioavailability of clopidogrel. Each rat received clopidogrel bisulfate (30 mg/kg) in DMSO/PEG400 (10/90, w/w) or 30 mg/kg of clopidogrel bisulfate in DMSO/PEG400/triethyl citrate (10.1/77.5/12.4, w/w/w). Serial plasma samples were obtained from each rat and concentrations of clopidogrel, its active drug, and clopidogrel acid in the plasma samples were determined by LC-MS/MS. It was found that in this study, concentrations of the active drug of clopidogrel were too low (<1 ng/ml) to be measured.

Table 4 lists the mean (n=3) values of AUC₀₋₂₄ₕᵣ, Cₘₐₓ and Tₘₐₓ of clopidogrel and clopidogrel acid. As evidenced from Table 4, triethyl citrate increases values of AUC₀₋₂₄ₕᵣ and Cₘₐₓ of clopidogrel by 171% and 156%, respectively. Thus, it is fair to conclude that triethyl citrate retards esterase hydrolysis and improves the absorption kinetics of clopidogrel bisulfate in rats.

TABLE 4
Mean (n = 3) Values of AUC_{0-24 hr}, Cₘₐₓ and Tₘₐₓ
of Clopidogrel and Clopidogrel Acid.

| | Dose | | | |
|---|---|---|---|---|
| | | | Clopidogrel Bisulfate + | |
| | Clopidogrel Bisulfate | | Triethyl Citrate | |
| | | Clopidogrel | | Clopidogrel |
| Parameter | Clopidogrel | Acid | Clopidogrel | Acid |
| AUC_{0-24 hr} | 28 | 12580 | 76 | 36810 |
| (ng · hr/ml) | | | | |
| Cₘₐₓ (ng/ml) | 7.3 | 2762 | 18.7 | 7851 |
| Tₘₐₓ (hr) | 4.1 | 8.0 | 6.0 | 8.0 |

In a rat study similar to the study described above, each rat received clopidogrel bisulfate (3 mg/kg) in DMSO/PEG400 (10/90, w/w) or 3 mg/kg of clopidogrel bisulfate in DMSO/PEG400/triethyl citrate/triacetin (10/78/6/6, w/w/w/w). Serial plasma samples were obtained from each rat and concentrations of clopidogrel and clopidogrel acid in the plasma samples were determined by LC-MS/MS.

Table 5 lists the mean (n=3) values of AUC₀₋₂₄ₕᵣ, Cₘₐₓ and Tₘₐₓ of clopidogrel and clopidogrel acid. As shown in Table 5, a combination of triethyl citrate and triacetin increases mean values of AUC₀₋₂₄ₕᵣ and Cₘₐₓ of clopidogrel by 650% and 638%, respectively. Thus, a combination of triethyl citrate and triacetin effectively improves the absorption kinetics of clopidogrel bisulfate in rats.

TABLE 5
Mean (n = 3) Values of AUC_{0-24 hr}, Cₘₐₓ and Tₘₐₓ
of Clopidogrel and Clopidogrel Acid.

| | Dose | | | |
|---|---|---|---|---|
| | | | Clopidogrel Bisulfate + | |
| | Clopidogrel Bisulfate | | Triethyl Citrate + Triacetin | |
| | | Clopidogrel | | Clopidogrel |
| Parameter | Clopidogrel | Acid | Clopidogrel | Acid |
| AUC₀₋₂₄ hr | 2 | 8616 | 15 | 4910 |
| (ng · hr/ml) | | | | |
| Cₘₐₓ (ng/ml) | 0.8 | 485 | 5.9 | 242 |
| Tₘₐₓ (hr) | 2.9 | 5.3 | 0.8 | 10 |

### Example 5

### Improvement of Absorption Kinetics of Capecitabine

Capecitabine is an ester prodrug, which is hydrolyzed mainly by CE2 in the GI track and CE1 in the liver and then converted by two enzymes to its active drug (5-fluorouracil) in the tumor after oral administration of capecitabine to cancer patients. A study similar to the clopidogrel study described above was conducted in rats (n=3) to ascertain the effect of triethyl citrate in improving the absorption kinetics and enhancing the bioavailability of capecitabine. Each rat received capecitabine (5 mg/kg) in DMSO or capecitabine (5 mg/kg) in DMSO/triethyl citrate (45 mg/kg). Serial plasma samples were obtained from each rat and concentrations of capecitabine in the plasma samples were determined by LC-MS/MS.

Table 6 lists the mean (n=3) values of AUC₀₋₂₄ₕᵣ, Cₘₐₓ and Tₘₐₓ of capecitabine. As shown in Table 6, triethyl citrate increases values of AUC₀₋₂₄ₕᵣ and Cₘₐₓ of capecitabine by 2,050% and 964%, respectively. Thus, triethyl citrate effectively retards esterase hydrolysis and improves the absorption kinetics of capecitabine in rats.

TABLE 6
Improvement of Absorption Kinetics of Capecitabine.

| | AUC_{0-24 hr} | Cₘₐₓ | Tₘₐₓ |
|---|---|---|---|
| Treatment | (ng · hr/ml) | (ng/ml) | (hr) |
| Capecitabine | 34 | 11.8 | 0.8 |
| Capecitabine + Triethyl Citrate | 731 | 125.5 | 8.5 |

In a rat study similar to the study described above, each rat received capecitabine (5 mg/kg) in DMSO or capecitabine (5 mg/kg) in DMSO/triethyl citrate (22.5 mg/kg)/triacetin (22.5 mg/kg). Serial plasma samples were obtained from each rat and concentrations of capecitabine in the plasma samples were determined by LC-MS/MS.

Table 7
Improvement of Absorption Kinetics of Capecitabine by a
Combination of Triethyl Citrate and Triacetin.

| | AUC_{0-24 hr} | Cₘₐₓ | Tₘₐₓ |
|---|---|---|---|
| Treatment | (ng · hr/ml) | (ng/ml) | (hr) |
| Capecitabine | 15 | 8.9 | 0.4 |
| Capecitabine + Triethyl Citrate + | 39 | 4.4 | 3.5 |
| Tracetin | | | |

Table 7 lists the mean (n=3) values of AUC₀₋₂₄ₕᵣ, Cₘₐₓ and Tₘₐₓ of capecitabine. As shown in Table 7, a combination of triethyl citrate and triacetin increases mean values of AUC₀₋₂₄ₕᵣ of capecitabine by 160%. Thus, a combination of triethyl citrate and triacetin effectively improves the absorption kinetics of capecitabine in rats.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements.

It will be understood that the above description of embodiments is given by way of example only and that various modifications may be made by those with ordinary skill in the art. The above specification, examples, and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of the present disclosure.

1. A method for improving the absorption of an ester prodrug in a subject comprising,
co-administering to the subject (1) an effective amount of the ester prodrug or a pharmaceutical acceptable salt thereof; and
(2) an adjuvant in an amount effective to impede a type I and/or type II carboxylase-mediated hydrolysis of the ester prodrug in the subject, wherein the adjuvant is selected from the group consisting of triacetin, triethyl citrate and a combination of both.

2. The method of claim 1, wherein the ester prodrug is an anti-coagulant, a DNA synthesis inhibitor, a topoisomerase 1 (TOP 1) inhibitor, an angiotensin II (AII) antagonist, an angiotensin-converting enzyme (ACE) inhibitor, an anti-thrombogenic agent, a peroxisome proliferator-activated receptor alpha (PPARα) agonist, a 3-hydroxy-3-methyl-glutaryl-coenzyme A (HMG-CoA) reductase inhibitor, an antibiotic, a reverse transcriptase inhibitor, a mitotic inhibitor, a neuraminidase inhibitor, an immunosuppressant, a gamma-aminobutyric acid (GABA) analogue, or a GABA_{B} receptor agonist.

3. (canceled)

4. (canceled)

5. The method of claim 2, wherein the anti-thrombogenic agent is clopidogrel, prasugrel, or aspirin.

6. The method of claim 2, wherein the anti-coagulant is dabigatran etexilate.

7. (canceled)

8. (canceled)

9. (canceled)

10. (canceled)

11. The method of claim 2, wherein the TOP 1 inhibitor is irinotecan.

12. The method of claim 2, wherein the DNA synthesis inhibitor is capecitabine.

13. The method of claim 2, wherein the neuraminidase inhibitor is oseltamivir or A-322278.

14. The method of claim 2, wherein the ester prodrug is clopidogrel, olmesartan medoxomil, tenofovir disoproxil, adefovir dipivoxil, mycophenolate mofetil, irinotecan, capecitabine, arbaclofen placarbil, dabigatran etexilate or gabapentin enacarbil.

15. In an improved pharmaceutical composition comprising an effective amount of an ester prodrug or a pharmaceutically acceptable salt thereof and a pharmaceutically acceptable excipient, wherein the improvement comprises,
an adjuvant selected from the group consisting of triacetin, triethyl citrate and a combination of both, and the adjuvant is present in an amount effective to impede a type I and/or type II carboxylase-mediated hydrolysis of the ester prodrug in vivo.

16. The improved pharmaceutical composition of claim 15, wherein the ester prodrug is clopidogrel, dabigatran etexilate, gabapentin enacarbil or capecitabine.

17. A method for impeding carboxylesterase-mediated hydrolysis of esters comprising contacting the carboxylesterase with triacetin, triethyl citrate, or both, in an amount effective to impede ester hydrolysis.
